(19) United States
(12) Patent Application Publication
(10) Pub. No.: US 2020/0410204 A1
(43) Pub. Date: Dec. 31, 2020

(54) CELL-IMAGE PROCESSING APPARATUS

(71) Applicant: OLYMPUS CORPORATION, Tokyo (JP)

(72) Inventor: Hitoshi ECHIGO, Kanagawa (JP)

(73) Assignee: OLYMPUS CORPORATION, Tokyo (JP)

(21) Appl. No.: 17/016,455

(22) Filed: Sep. 10, 2020

Related U.S. Application Data
(63) Continuation of application No. PCT/JP2018/010202, filed on Mar. 15, 2018.

(51) Int. Cl. G06K 9/00; G06T 7/00; G01N 33/483

(52) U.S. Cl. CPC G06K 9/0014; G06T 7/0012; G06T 2207/30024; G01N 33/4833; G06K 9/00134

(57) ABSTRACT

A cell-image processing apparatus includes: a processor including hardware; and a storage, wherein the processor is configured to: extract, regarding a plurality of images acquired by capturing, over time, images of cells that are being cultured, a plurality of common measurement regions among the images; and calculate proliferation speeds of the cells contained in the respective extracted measurement regions. The storage is configured to store the calculated proliferation speeds and positional information of the respective measurement regions in association with each other.

### CROSS-REFERENCE TO RELATED APPLICATIONS

This is a continuation of International Application PCT/JP2018/010202, with an international filing date of Mar. 15, 2018, which is hereby incorporated by reference herein in its entirety.

### TECHNICAL FIELD

The present invention relates to a cell-image processing apparatus.

### BACKGROUND ART

In the process of preparing pluripotent cells such as ES cells and iPS cells, cells that do not have pluripotent cell properties occur in a large number as a result of failing to introduce or express genes. In order to employ pluripotent cells in regenerative medicine or the like, it is necessary to remove the cells that do not have pluripotent cell properties and to extract only the pluripotent cells.

For example, in order to determine whether or not iPS cells have been formed, there is a known method in which a protein known as an undifferentiated marker, such as Oct3/4, Nanog, TRA-1-60, or TRA-1-81, that is expressed in cells having pluripotency is detected by means of qPCR or immunostaining (for example, see Patent Literature 1).

### CITATION LIST

### Patent Literature

### {PTL 1} Japanese Unexamined Patent Application, Publication No. 2014-100141

### SUMMARY OF INVENTION

An aspect of the present invention is directed to a cell-image processing apparatus including: a processor comprising hardware; and a storage, wherein the processor is configured to: extract, regarding a plurality of images acquired by capturing, over time, images of cells that are being cultured, a plurality of common measurement regions among the images; and calculate proliferation speeds of the cells contained in the respective extracted measurement regions, and wherein the storage is configured to store the calculated proliferation speeds and positional information of the respective measurement regions in association with each other.

### Advantageous Effects of Invention

The present invention affords an advantage in that it is possible to efficiently extract specific cells that are present in a culture vessel.

### BRIEF DESCRIPTION OF DRAWINGS

FIG. 1 is a block diagram showing a cell-image processing apparatus according to an embodiment of the present invention.

FIG. 2 is a diagram showing examples of measurement regions extracted by the cell-image processing apparatus in FIG. 1.

FIG. 3 is a diagram showing an example of a display in which proliferation speeds and the positional information are displayed in association with each other by means of the cell-image processing apparatus in FIG. 1.

FIG. 4 is a diagram showing another example of a display that is similar to FIG. 3.

FIG. 5 is a diagram showing another example of a display that is similar to FIG. 3.

FIG. 6 is a diagram showing a graph of changes over time in the number of cells in a specified specific measurement region.

FIG. 7 is a diagram showing examples of an entire image, a video image, and a graph of changes over time in the number of cells simultaneously displayed by the cell-image processing apparatus in FIG. 1

FIG. 8 is an overall configuration diagram showing an observation apparatus that acquires an image.

FIG. 9 is a perspective view showing a portion of an illumination optical system in the observation apparatus in FIG. 8.

FIG. 10A is a side view showing an example of a line light source in the illumination optical system in FIG. 9.

FIG. 10B is a front view in which the line light source in FIG. 10A is viewed in the optical axis direction.

FIG. 11 is a diagram showing another example of the line light source in the illumination optical system in FIG. 9.

FIG. 12 is a diagram showing an objective optical system group in the observation apparatus in FIG. 8.

FIG. 13 is a diagram showing arraying of objective optical systems in the objective optical system group in FIG. 12.

FIG. 14 is a diagram showing arraying of aperture stops in the objective optical system group in FIG. 12.

FIG. 15 is a diagram showing the arrangement of a line sensor at an image surface of the objective optical system group in FIG. 12.

FIG. 16 is a diagram showing the arrangement of the line light source, a cylindrical lens, and a prism in the illumination optical system in FIG. 9.

FIG. 17 is a diagram for explaining the effect of oblique illumination.

FIG. 18 is a diagram showing an example of an image of a sample illuminated by the oblique illumination in FIG. 17.

FIG. 19A is a diagram showing an example of the sample.

FIG. 19B is a diagram showing two-dimensional images of the sample in FIG. 19A acquired by the observation apparatus in FIG. 8.

FIG. 19C is a diagram showing an image acquired by applying inversion processing and joining processing to the images in FIG. 19B.

FIG. 20 is a diagram showing the arrangement of the line light source in another aspect of the observation apparatus in FIG. 8.

### DESCRIPTION OF EMBODIMENT

A cell-image processing apparatus 51 according to an embodiment of the present invention will be described below with reference to the drawings.

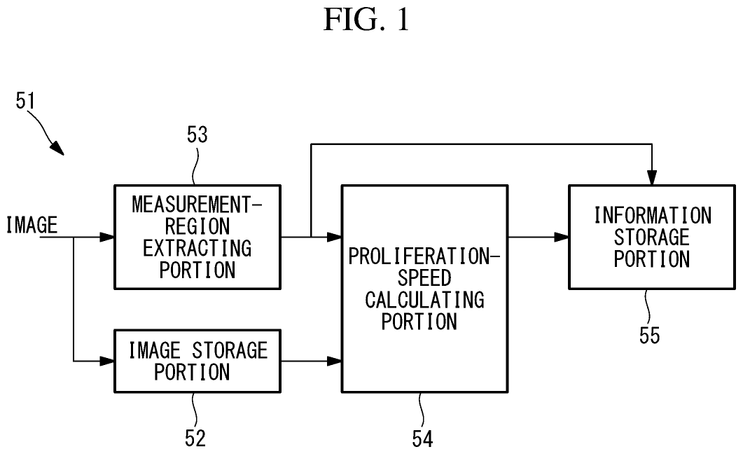

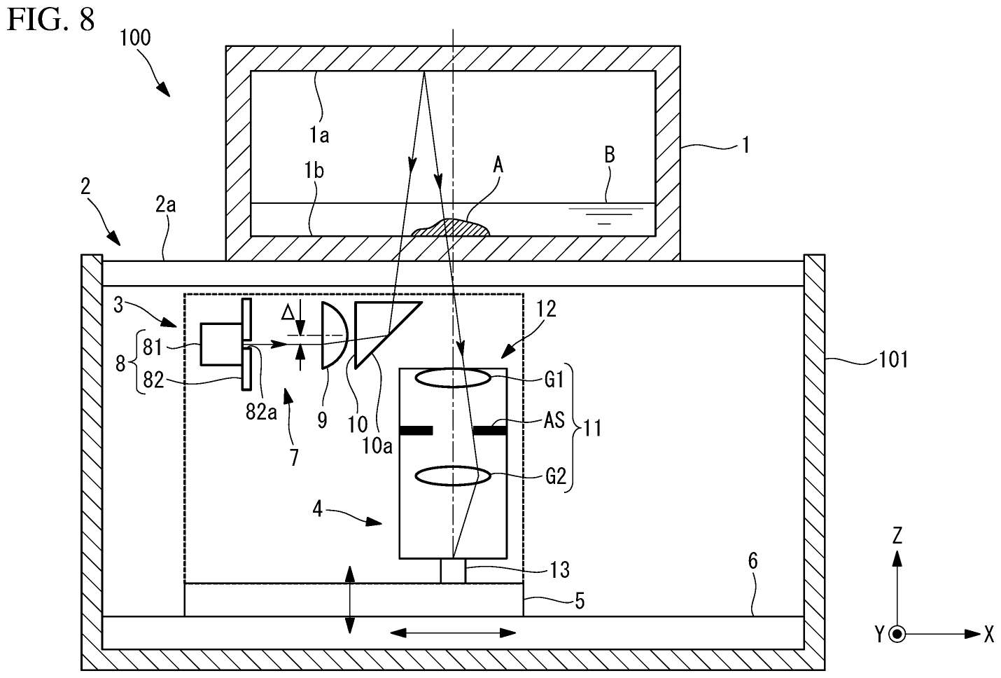

The cell-image processing apparatus 51 according to this embodiment is an apparatus that processes a plurality of images acquired by an observation apparatus (see FIG. 8) 100, and includes, as shown in FIG. 1: an image storage portion 52 that stores images that are input from the observation apparatus 100 in a time series; a measurement-region extracting portion 53 that extracts a plurality of common measurement regions among the images; a proliferation-speed calculating portion 54 that calculates the proliferation speed of cells contained in each of the extracted measurement regions; and an information storage portion (storage portion) 55 that stores the calculated proliferation speeds and positional information of the respective measurement regions in association with each other. The measurement-region extracting portion 53 and the proliferation-speed calculating portion 54 are constituted of processors, and the image storage portion 52 and the information storage portion 55 are constituted of memories, storage media, or the like.

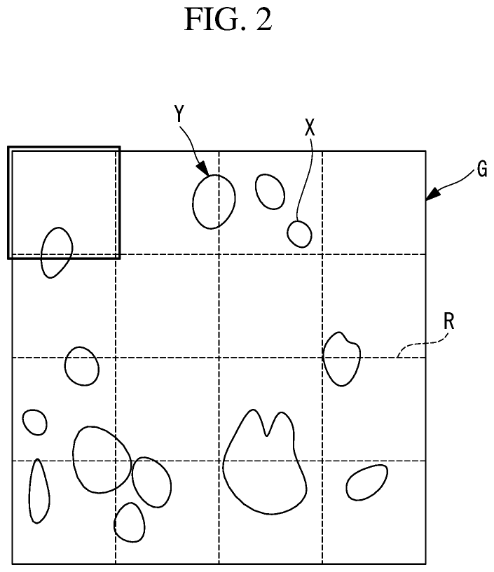

The measurement-region extracting portion 53 sets a plurality of measurement regions in one of the images input from the observation apparatus 100, and extracts measurement regions that are common with the set measurement regions in another image input from the observation apparatus 100. For example, as shown in FIG. 2, a plurality of measurement regions R formed of rectangular shapes of equal sizes may be set by dividing an image G with reference to edges of the image G, and the common measurement regions R formed of rectangular shapes of equal sizes may also be extracted in another image G in a similar manner with reference to the edges of the image G.

As the other image, an image G that is temporally close to the image G in which the measurement regions R are set, for example, an image G acquired before or after said image G, is selected.

The proliferation-speed calculating portion 54 measures the number of cells in the common measurement regions R in the two or more selected images G, and calculates the proliferation speed for each of the measurement regions R in the form of the amount of change in the number of cells per unit time.

First, by means of edge detection or outline tracking in the measurement regions R, boundaries between cells X and areas other than the cells X are extracted, areas having closed boundaries are recognized as the cells X or colonies Y, and the cells X and the colonies Y are distinguished from each other on the basis of the sizes thereof.

Then, those recognized as the cells X are counted to obtain the number of cells. Regarding those recognized as the colonies Y, the areas thereof are calculated from the number of pixels in said colonies Y, and the number of cells therein is calculated by dividing said areas by the average area of a single cell X. All of the cells X and the number of cells in the colonies Y in the measurement regions R are added up to calculate the number of cells in the measurement regions R.

It is possible to calculate the proliferation speeds by dividing the differences in the number of cells calculated for all of the common measurement regions R between the two images G by the time difference between said images G.

The information storage portion 55 stores the coordinates (positional information) of representative points of the extracted measurement regions R and the proliferation speeds calculated for said measurement regions R in association with each other.

The operation of the thus-configured cell-image processing apparatus 51 according to this embodiment will be described below.

When the observation apparatus 100 acquires two images G of a culture surface in a vessel (culture vessel) 1 with a prescribed time interval therebetween, the acquired images G are transmitted to the cell-image processing apparatus 51.

With the cell-image processing apparatus 51 according to this embodiment, the measurement regions R are set in one of the transmitted images G, and measurement regions R that are common with the set measurement regions R are extracted in the other image G. In other words, in the two images G, the plurality of corresponding measurement regions R are set.

Then, regarding each of the set measurement regions R, the proliferation-speed calculating portion 54 calculates the proliferation speed, and the calculated proliferation speed and the positional information of the measurement region R having said proliferation speed are stored in the information storage portion 55 in association with each other.

Therefore, in the case in which an observer observes the image G and confirms the presence of specific cells X in one measurement region R in the image G, it is possible to selectively observe cells X having characteristics that are equivalent to those of the confirmed specific cells X by observing measurement regions R that are associated with proliferation speeds that are equivalent to the proliferation speed in said measurement region R.

In other words, as compared with the case in which all of points in the images G are observed one by one, it is possible to perform observation by sorting the cells X having characteristics that are equivalent to those of the specific cells X targeted for observation, and it is possible to sort the specific cells X in a manner in which the required amounts of time and labor are reduced. In addition, because the specific cells X are sorted on the basis of the proliferation speeds, there is an advantage in that it is possible to sort the cells X in a precise manner even in the case in which the sizes of colonies Y are small.

Note that, although this embodiment goes only so far as to store the proliferation speeds and the positional information of the measurement regions R in the information storage portion 55 in association with each other, this embodiment may include a display portion that displays the proliferation speeds and the positional information of the measurement regions R stored in the information storage portion 55 in association with each other.

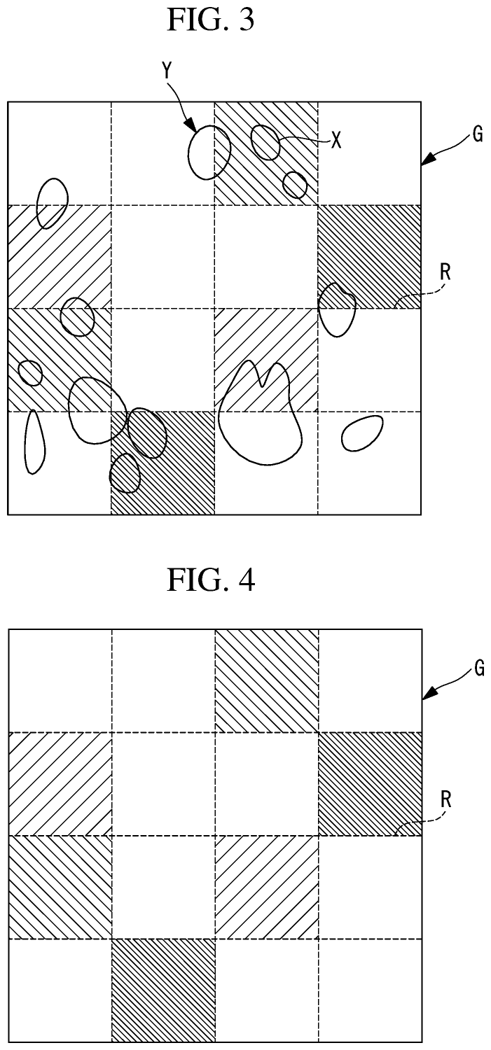

For example, as shown in FIG. 3, the display portion may display one of the images G transmitted from the observation apparatus 100, and may display, so as to be superimposed on the image G, measurement regions R that are color-coded in accordance with the proliferation speeds. In addition, as shown in FIG. 4, only the color-coded measurement regions R may be displayed without being superimposed on the image G. By doing so, it is possible to display the proliferation speeds for separate measurement regions R in the form of a heat map.

In addition, in this embodiment, the measurement-region extracting portion 53 sets the plurality of measurement regions R formed of rectangular shapes having equal sizes by dividing the images G with reference to the edges of the images G; however, alternatively, a plurality of measurement regions R formed of rectangular shapes having arbitrary sizes may be set at arbitrary positions on the images G. In this case, the cells X and the colonies Y at close positions between the images G may be estimated as being in the same regions. In addition, the common measurement regions R may be extracted by performing matching processing between the images G. In addition, the measurement regions R may be extracted with reference to the vessel or labels captured in the images G.

In addition, the colonies Y themselves may be extracted as the measurement regions R.

In this case, by recognizing boundaries by means of edge detection or outline tracking, areas having closed boundaries may be recognized as cells X or colonies Y, and the cells X and the colonies Y may be distinguished from each other on the basis of the sizes thereof.

In this case also, the cells X and colonies Y at close positions between the images G may be estimated as being in the same regions. In addition, the common measurement regions R may be extracted by performing matching processing between the images G.

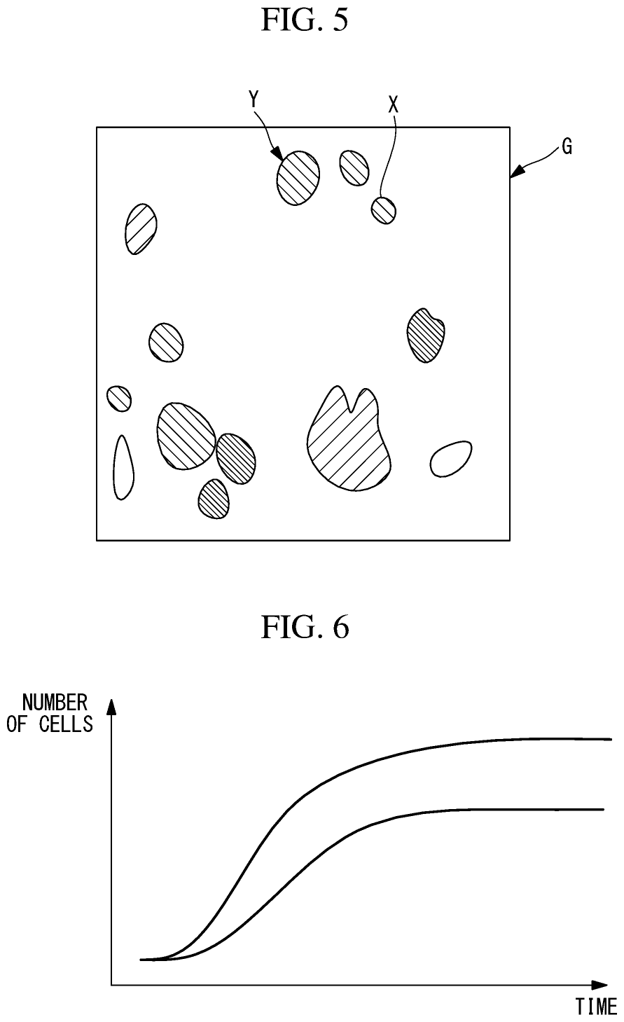

In this case, the proliferation speed is calculated for each of the colonies Y, and, as shown in FIG. 5, the colonies Y are displayed by color-coding each of the colonies Y according to the proliferation speeds; therefore, there is an advantage in that the observer can perform observation by more efficiently sorting the cells X.

In addition, in the case in which the colonies Y themselves are used as the measurement regions R, whether or not set the colonies Y as the measurement regions R may be determined from a plurality of parameters, such as area, shape, texture, and so forth of the colonies Y. By doing so, as measurement targets, it is possible to set only colonies Y that fit the purpose.

In addition, the selection standards may be changed in accordance with the types of cells X or the purpose of culturing. By doing so, it is possible to select colonies Y that fit the purpose. In this case, by including a table of the selection standards, it is possible to easily switch the selection standards. In addition, the selection standards may be set, as appropriate, by the observer.

In addition, colonies Y in which a plurality of colonies Y are combined, colonies Y formed from a plurality of cell types, or regions that do not contain any colony Y may be excluded in advance from an area in which the measurement regions R would be set.

In addition, when the observer specifies one of the measurement regions R in one of the images G, as shown in FIG. 6, a graph showing the change over time in the number of cells in the specified measurement region R may be displayed.

Furthermore, when the observer specifies one of the colonies Y in one of the images (entire images) G, a video image showing the change over time in the measurement region R containing the specified colony Y may be displayed. In other words, in the case in which a colony Y is specified in one of the entire images G, partial images H, which contain the corresponding colonies Y in a plurality of entire images of the past and future with reference to the entire image G in which the colony Y is specified, may be cut out and said images may be sequentially displayed from the older images by switching among the images at prescribed time intervals.

In the case in which the specified colony Y is displayed as a video image, a center position of the colony Y may be calculated, and the partial images may be cut out from the entire images G in the area in which the center positions of the colonies Y extracted in the respective images G are at the center of the video image. This reduces blurring due to movement of the colony Y while replaying the video image, and thus, it is possible to facilitate visual recognition of the change over time in the colony Y.

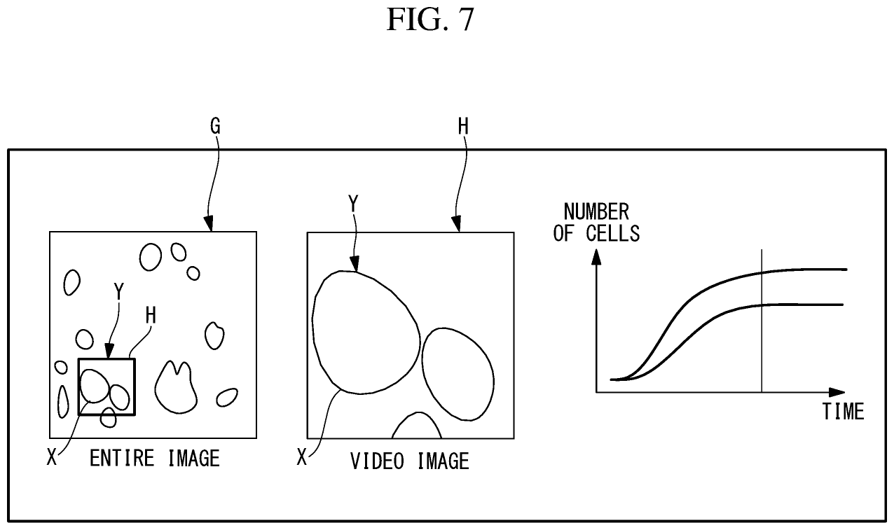

In addition, in the case in which the video image of the colony Y is displayed, as shown in FIG. 7, it is preferable to simultaneously display: the entire images G in which the partial images H containing the specified colony Y are displayed; the video image of the partial images H cut out from the entire images G; and the graph showing the change over time in the proliferation of the cells X in the specified colony Y. In the graph showing the change over time in the proliferation of the cells X, it is preferable to display a time display representing the times of the displayed video image, for example, a straight line (or an arrow or the like), and the times of the displayed video image may be changed by sliding the time display in the time axis direction.

In addition, when storing the proliferation speeds and the positional information of the measurement regions R in the information storage portion 55 in association with each other, the measurement regions R in which the proliferation speeds are close to each other may be grouped and stored. In this case, the measurement regions R in which the proliferation speeds are close to each other may be grouped on the basis of cluster analysis, which is one statistical method, or boundary values that are set in advance.

Here, an example of the observation apparatus 100 for acquiring the entire image G of the cells X will be described.

As shown in FIG. 8, the observation apparatus 100 includes: a stage 2 that supports the vessel 1 accommodating a sample A; an illumination portion 3 that radiates illumination light onto the sample A supported by the stage 2; an image acquisition portion 4 that acquires an image G of the sample A by detecting the illumination light that has passed through the sample A by means of a line sensor 13; a focus adjusting mechanism 5 that adjusts the focal point of the image acquisition portion 4 with respect to the sample A; and a scanning mechanism 6 that moves the image acquisition portion 4 in a scanning direction that is orthogonal to a longitudinal direction of the line sensor 13. The illumination portion 3, the image acquisition portion 4, the focus adjusting mechanism 5, the scanning mechanism 6, and the line sensor 13 are accommodated, in an airtight state, in a housing 101 in which a top face is closed by the stage 2.

In the following description, an XYZ Cartesian coordinate system will be employed, wherein the direction along an optical axis of the image acquisition portion 4 (the optical axis of an objective optical system 11) is the Z-direction, the scanning direction of the image acquisition portion 4 due to the scanning mechanism 6 is the X-direction, and the longitudinal direction of the line sensor 13 is the Y-direction. As shown in FIG. 8, the observation apparatus 100 is disposed in an orientation in which the Z-direction corresponds to a vertical direction and the X-direction and the Y-direction correspond to horizontal directions.

The vessel 1 is a vessel, such as a cell-culturing flask or dish, formed from an optically transparent resin as a whole, and has a top plate 1a and a bottom plate 1b that face each other. The sample A is, for example, cells that are cultured in a medium B. An inner surface of the top plate 1a is a reflective surface at which Fresnel reflection of the illumination light occurs.

The stage 2 includes a flat-plate-like mounting base 2a that is horizontally disposed, and the vessel 1 is placed on the mounting base 2a. The mounting base 2a is formed from an optically transparent material, for example, glass, so as to allow the illumination light to pass therethrough.

The illumination portion 3 includes an illumination optical system 7 that is disposed below the stage 2 and that emits line-like illumination light obliquely upward, and radiates the illumination light onto the sample A from obliquely thereabove as a result of the illumination light being reflected obliquely downward at the top plate (reflective member) la.

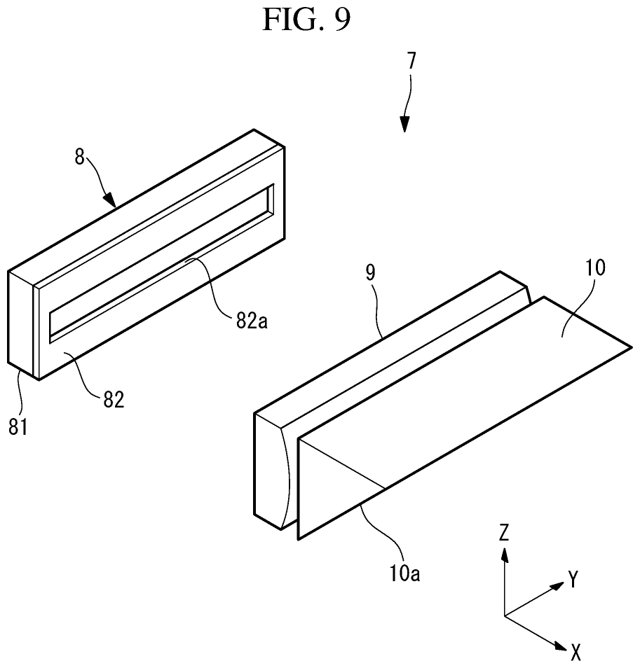

Specifically, as shown in FIG. 9, the illumination optical system 7 includes: a line light source 8 that is disposed at a side of the image acquisition portion 4 and that emits the illumination light in the X-direction toward the image acquisition portion 4; a cylindrical lens (lens) 9 that converts the illumination light emitted from the line light source 8 to a collimated light beam; and a prism (deflection element) 10 that deflects upward the illumination light emitted from the cylindrical lens 9.

The line light source 8 includes: a light-source body 81 having an emitting surface from which light is emitted; and an illumination mask 82 that is provided on the emitting surface of the light-source body 81. The illumination mask 82 has a rectangular opening 82a having a short side that extends in the Z-direction and a long side that extends in the Y-direction and that is longer than the short side. As a result of the light emitted from the emitting surface passing through only the opening 82a, illumination light that has a line-like lateral cross-section (cross-section that intersects the optical axis of the illumination light) in which the longitudinal direction thereof corresponds to the Y-direction is generated.

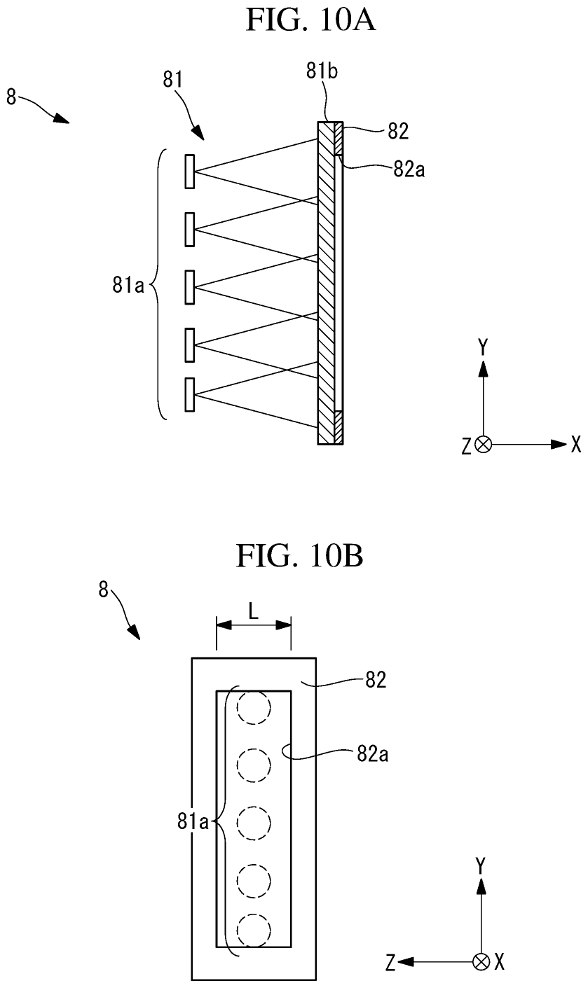

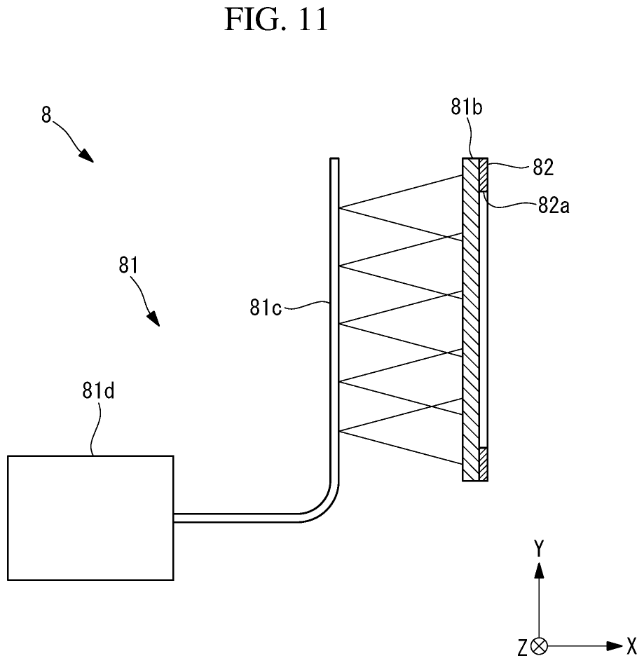

FIGS. 10A, 10B, and 11 show examples of specific configurations of the line light source 8.

In the line light source 8 in FIGS. 10A and 10B, the light-source body 81 includes: an LED array 81a formed from LEDs that are arrayed in a single row in the Y-direction; and a diffusion plate 81b that diffuses light emitted from the LED array 81a. The illumination mask 82 is provided on an emitting-side surface of the diffusion plate 81b.

In the line light source 8 in FIG. 11, the light-source body 81 includes: a light-diffusing optical fiber 81c; and a light source 81d, such as an LED or an SLD (superluminescent diode) that supplies light to the optical fiber 81c. As a result of employing the light-diffusing optical fiber 81c, it is possible to increase the evenness of the illumination light intensity as compared to the case in which the LED array 81a is employed.

The cylindrical lens 9 has a curved surface that extends in the Y-direction and that is curved only in the Z-direction on the opposite side of the line light source 8. Therefore, the cylindrical lens 9 has refractive power in the Z-direction and does not have refractive power in the Y-direction. In addition, the illumination mask 82 is positioned at a focal surface of the cylindrical lens 9 or in the vicinity of the focal surface. Accordingly, the illumination light emitted from the opening 82a of the illumination mask 82, which is in the form of divergent light beams, is bent by the cylindrical lens 9 only in the Z-direction, and thus, said light is converted to light beams that have a certain dimension in the Z-direction (collimated light beam in the XZ-plane).

The prism 10 has a deflection surface 10a that is inclined at a 45° angle with respect to the optical axis of the cylindrical lens 9 and that deflects the illumination light that has passed through the cylindrical lens 9 upward. The illumination light that has been deflected at the deflection surface 10a passes through the mounting base 2a and the bottom plate 1b of the vessel 1 and illuminates the sample A from above by being reflected at the top plate 1a, and the illumination light that has passed through the sample A and the bottom plate 1b enters the image acquisition portion 4.

The image acquisition portion 4 includes: an objective optical system group 12 having a plurality of objective optical systems 11 that are arrayed in a single row; and the line sensor 13 that captures an optical image of the sample A formed by the objective optical system group 12.

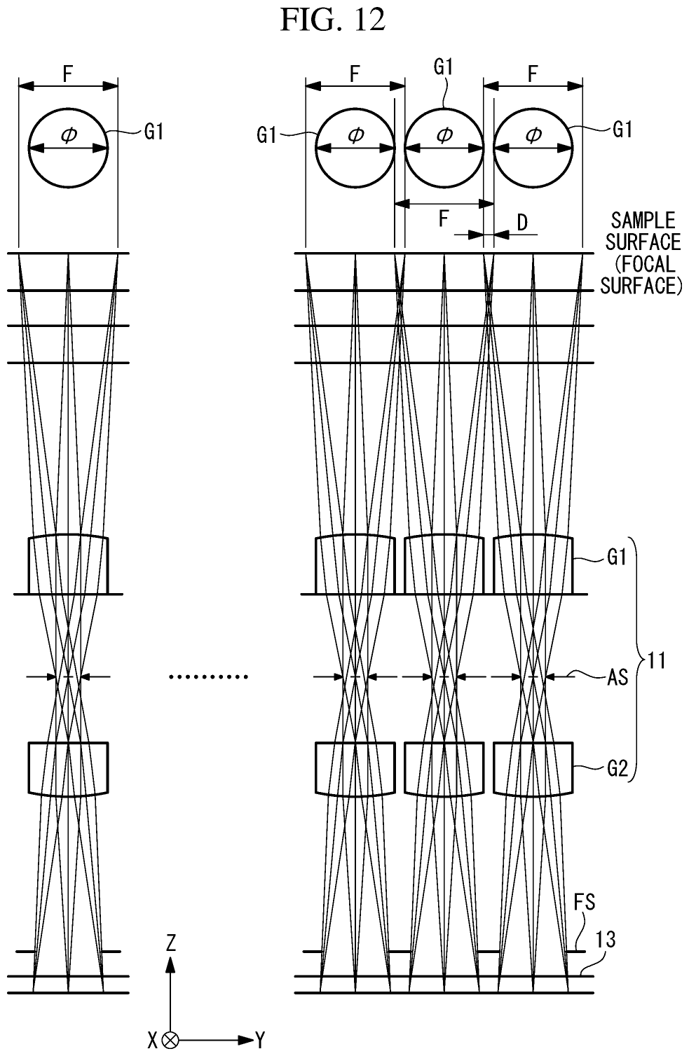

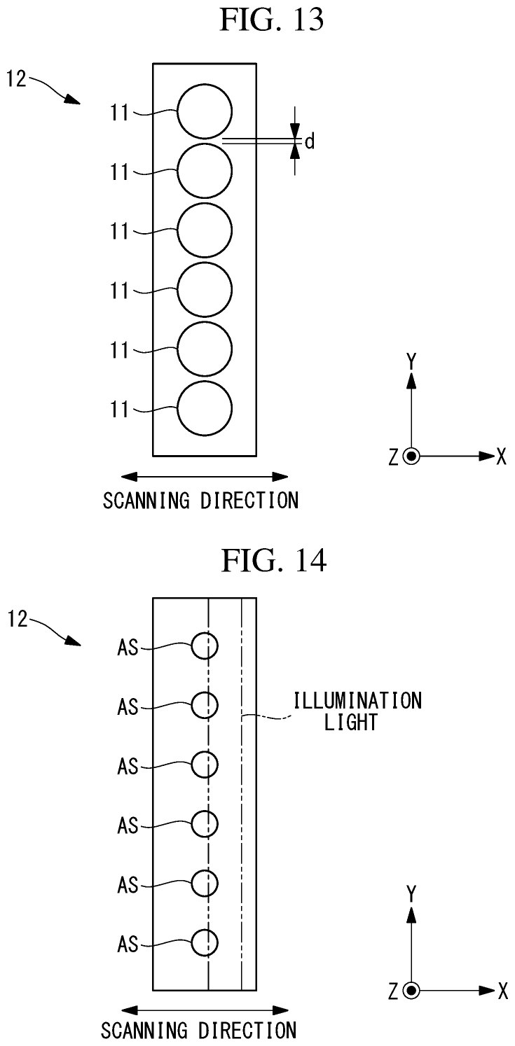

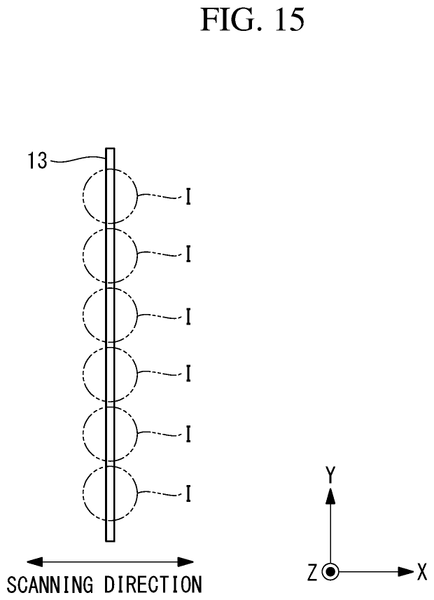

As shown in FIG. 12, each of the objective optical systems 11 includes a first lens group G1, an aperture stop AS, and a second lens group G2, in this order from the object side (sample A side). As shown in FIG. 13, the plurality of objective optical systems 11 are arrayed in the Y-direction so that the optical axes thereof extend in the Z-direction so as to be parallel to each other and so that optical images are formed on the same surface. Therefore, a plurality of optical images I that are arranged next to each other in a single row in the Y-direction are formed at the image surface (see FIG. 15). As shown in FIG. 14, the aperture stops AS are also arrayed in a single row in the Y-direction.

The line sensor 13 has a plurality of light-receiving elements that are arrayed in a longitudinal direction, and acquires a line-like one-dimensional image. As shown in FIG. 15, the line sensor 13 is disposed in the Y-direction over the image surface of the plurality of objective optical systems 11. The line sensor 13 acquires a line-like one-dimensional image of the sample A by detecting the illumination light that has formed the optical images I at the image surface.

Gaps d are formed between the adjacent objective optical systems 11. In order to obtain an image that has no break in the image of the sample A in the Y-direction, the objective optical system group 12 satisfies the following two conditions.

The first condition is that, in each of the objective optical systems 11, the entrance pupil position is at a position that is farther on the image side than the first lens group G1, which is positioned closest to the sample A side, is, as shown in FIG. 12. This is realized by disposing the aperture stops AS farther on the object side than the image-side focal point of the first lens group G1 is. As a result of satisfying the first condition, off-axis rays approach the optical axes of the objective optical systems 11 when approaching the first lens group G1 from the focal surface; therefore, a real viewing field F in the direction perpendicular to the scanning direction (Y-direction) becomes greater than a diameter φ of the first lens group G1. Therefore, the viewing fields of two adjacent objective optical systems 11 overlap with each other in the Y-direction, and thus, an optical image of the sample A having no break in the viewing fields is formed at the image surface.

The second condition is that, as shown in FIG. 12, the absolute value of projected lateral magnification between the object surface of each of the objective optical systems 11 and the image surface is one or less. As a result of satisfying the second condition, the plurality of optical images I formed by the plurality of objective optical systems 11 are arrayed in the Y-direction at the image surface without overlapping with each other. Therefore, the line sensor 13 can capture, in a mutually spatially separated manner, the plurality of optical images I formed by means of the plurality of objective optical systems 11. In the case in which the projected lateral magnification is greater than one, the two adjacent optical images I overlap with each other in the Y-direction at the image surface.

Even in the case in which the second condition is satisfied, it is preferable to provide, in the vicinity of the image surface, a field stop FS that restricts the area through which the illumination light passes in order to reliably prevent light traveling outside farther than the real viewing field F from overlapping in the adjacent optical images.

An example of the objective optical system group 12 will be indicated below.

The entrance pupil position (distance to the entrance pupil from a surface that is closest to the object side of the first lens group G1): 20.1 mm

The projected lateral magnification: ×−0.756

The real viewing field F: 2.66 mm

The lens diameter φ of the first lens group G1: 2.1 mm

The Y-direction lens interval d of the first lens group G1: 2.3 mm

The overlapping width D of the viewing field: 0.36 mm (=2.66/2−(2.3−2.66/2))

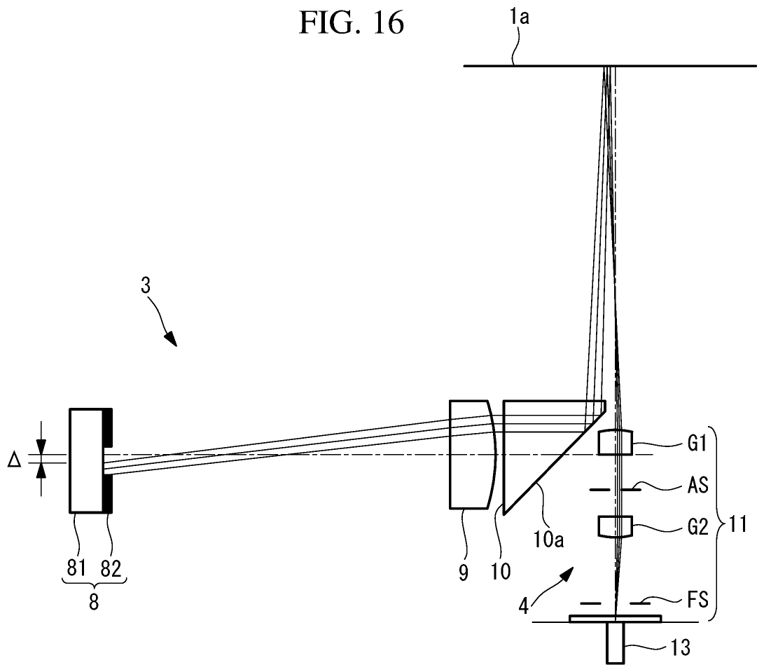

Here, the illumination portion 3 is configured so as to perform oblique illumination in which the illumination light is radiated onto the sample A in an oblique direction with respect to the optical axis of the image acquisition portion 4. Specifically, as shown in FIG. 16, the illumination mask 82 is positioned at or in the vicinity of the focal surface of the cylindrical lens 9, as described above, and the center of the short side of the illumination mask 82 is eccentrically disposed on the bottom side with respect to the optical axis of the cylindrical lens 9 by a distance A. Accordingly, the illumination light is emitted from the prism 10 in a direction that is inclined with respect to the Z-direction in the XZ-plane. Then, the illumination light reflected at the substantially horizontal top plate 1a obliquely enters the sample surface (focal surfaces of the objective optical systems 11) with respect to the Z-direction in the XZ-plane, and the illumination light that has passed through the sample A obliquely enters the objective optical systems 11.

Because the illumination mask 82 has width in the short-side direction, the illumination light that has been converted to the collimated light beam by the cylindrical lens 9 has an angle distribution. When such illumination light obliquely enters the objective optical systems 11, only a portion thereof positioned on the optical axis side passes through the aperture stops AS and reaches the image surface, as indicated by the two-dot chain line in FIG. 14, and other portions positioned outside with respect to the optical axis are blocked by outer edges of the aperture stops AS.

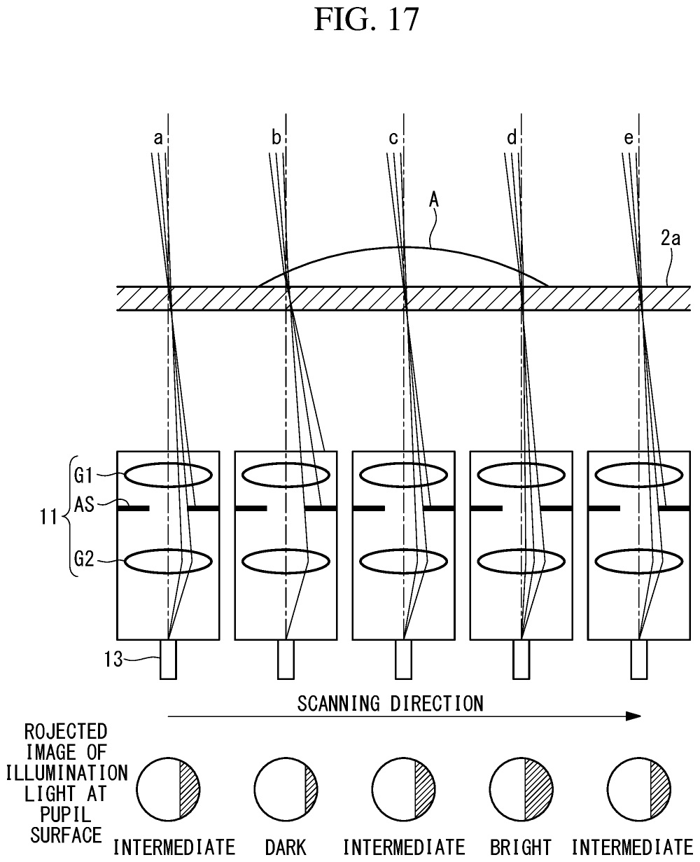

FIG. 17 is a diagram for explaining the effect of the oblique illumination when observing, as the sample A, cells having a high refractive index. The objective optical systems 11 are assumed to be moved from left to right in FIG. 17. In the case in which the entry angle of the illumination light is equivalent to the capture angles of the objective optical systems 11, light rays a and e that have passed through regions in which the sample A is absent and a light ray c that has substantially vertically entered the surface of the sample A pass through the vicinity of a peripheral edge of the entrance pupil with nearly no refraction and reach the image surface. Such light rays a, c, and e form optical images of intermediate brightness at the image surface.

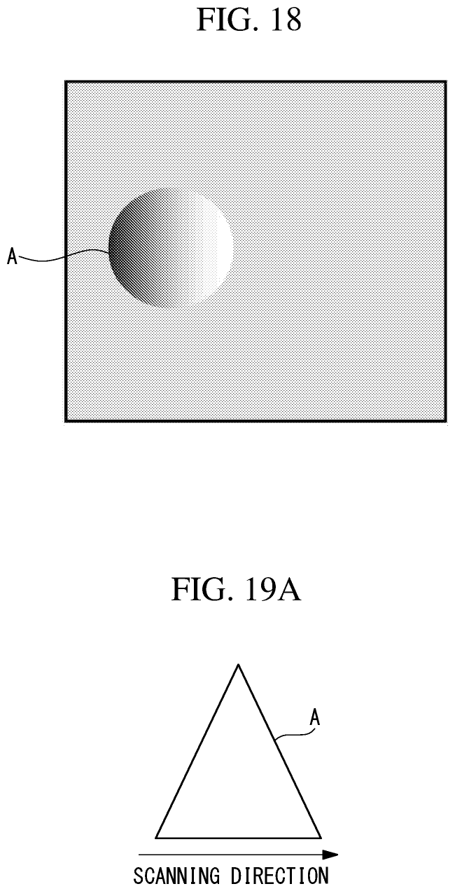

A light ray b that has passed through a left end of the sample A in FIG. 17 is refracted outward, reaches outside the entrance pupil, and is vignetted by the aperture stops AS. Such a light ray b forms a dark optical image at the image surface. A light ray d that has passed through a right end of the sample A in FIG. 17 is refracted inward, and passes through the sample A farther inside than the peripheral edge of the entrance pupil. Such a light ray d forms a brighter optical image at the image surface. As a result of the above-described image formation, as shown in FIG. 18, a high-contrast image of the sample A, in which one side thereof is bright and a shadow is formed on the other side, thus having a three-dimensional appearance, is acquired.

In order for the illumination light to have such an angle distribution that a portion of the illumination light that has obliquely entered the objective optical systems 11 passes through the aperture stops AS and other portions thereof are blocked by the aperture stops AS, it is preferable that, when the illumination light enters the objective optical systems 11, the entry angles of the illumination light with respect to the optical axes of the objective optical systems 11 satisfy the Conditional Expressions (1) and (2) below:

θmin>0.5NA (1); and

θmax<1.5NA (2).

θmin is a minimum value of the entry angles of the illumination light with respect to the optical axes of the objective optical systems 11 (entry angle of the light ray positioned closest to the optical axis side), θmax is a maximum value of the entry angles of the illumination light with respect to the optical axes of the objective optical systems 11 (entry angle of the light ray positioned at the outermost side in the radial direction with respect to the optical axis), and NA is the numerical aperture of the objective optical systems 11.

It is experimentally confirmed that the images G of the sample A having high contrast are acquired when Conditional Expressions (1) and (2) are satisfied in observation performed by using the observation apparatus 100. In order to satisfy Conditional Expressions (1) and (2), it is preferable that a focal distance F1 of the cylindrical lens 9 and a length L of the short side of the opening 82a of the illumination mask 82 satisfy Conditional Expression (3) below:

L>(θmax−θmin)F1 (3)

Furthermore, in the case in which the deflection angle of the prism 10 (the inclination angle of the deflection surface 10a with respect to the optical axes of the objective optical systems 11) is 45°, it is preferable that an amount of shift (eccentric distance) A of the center position of the short side of the illumination mask 82 with respect to the optical axis of the cylindrical lens 9 satisfy Conditional Expression (4) below:

Δ=NA/F1 (4)

In the case in which the deflection angle of the prism 10 is not 45°, A is corrected in accordance with the amount by which the deflection angle deviates from 45°. Specifically, Δ is increased in the case in which the deflection angle is greater than 45°, and Δ is decreased in the case in which the deflection angle is less than 45°.

As a result of satisfying Conditional Expressions (1)-(4), it is possible to acquire images G having a high contrast even if the sample A includes phase objects such as cells. In the case in which Conditional Expressions (1)-(4) are not satisfied, the contrast of the sample A decreases.

The focus adjusting mechanism 5 integrally moves, for example, by means of a linear actuator (not shown), the illumination optical system 7 and the image acquisition portion 4 in the Z-direction. Accordingly, it is possible to change the Z-direction positions of the illumination optical system 7 and the image acquisition portion 4 with respect to the still stage 2, and thus, it is possible to focus the objective optical system group 12 with respect to the sample A.

The scanning mechanism 6 moves, for example, by means of a linear actuator that supports the focus adjusting mechanism 5, the image acquisition portion 4 and the illumination optical system 7 integrally with the focus adjusting mechanism 5 in the X-direction.

Note that the scanning mechanism 6 may be configured with a system that moves the stage 2 in the X-direction instead of the image acquisition portion 4 and the illumination optical system 7, or may be configured so that the image acquisition portion 4, the illumination optical system 7, and the stage 2 can all be moved in the X-direction.

Next, the operation of the observation apparatus 100 will be described using, as an example, the case of observing the sample A, which is the cells being cultured in the vessel 1.

The line-like illumination light emitted in the X-direction from the line light source 8 is converted to a collimated light beam by the cylindrical lens 9, is deflected upward by the prism 10, and is emitted obliquely upward with respect to the optical axis. The illumination light passes through the mounting base 2a and the bottom plate 1b of the vessel 1, is reflected obliquely downward at the top plate 1a, passes through the sample A, the bottom plate 1b, and the mounting base 2a, and is focused by the plurality of objective optical systems 11. The illumination light that obliquely travels inside each of the objective optical systems 11 is partially vignetted at the aperture stops AS, and only a portion thereof passes through the aperture stops AS; consequently, the illumination light forms a shadowed optical image of the sample A at the image surface.

The optical image of the sample A formed at the image surface is acquired by the line sensor 13 disposed at the image surface, and thus, one-dimensional image of the sample A is acquired. The image acquisition portion 4 repeats the acquisition of the one-dimensional image by means of the line sensor 13 while being moved in the X-direction by means of the operation of the scanning mechanism 6. By doing so, a two-dimensional image of the sample A distributed over the bottom plate 1b is acquired.

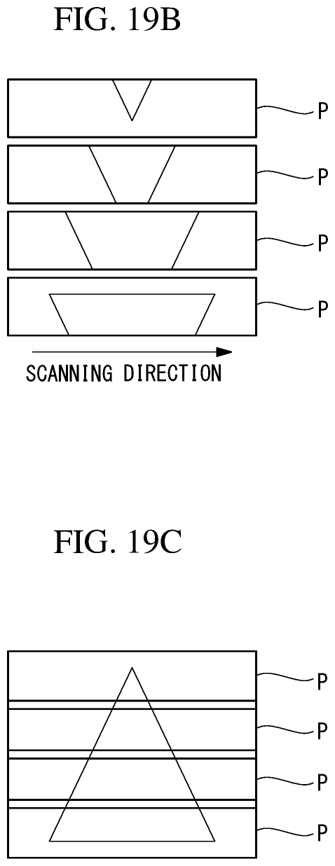

Here, the images formed by the respective objective optical systems 11 at the image surface are inverted images. Therefore, for example, in the case in which the two-dimensional image of the sample A shown in FIG. 19A is acquired, the images are inverted in partial images P corresponding to the respective objective optical systems 11, as shown in FIG. 19B. In order to correct the inverted images, processing in which the individual partial images P are turned in a direction that is perpendicular to the scanning direction is performed, as shown in FIG. 19C.

In the case in which the absolute value of the projected lateral magnifications of the objective optical systems 11 is greater than one, the viewing fields at edge portions of the individual partial images P overlap with the viewing fields at the edge portions of the adjacent partial images P. In this case, as shown in FIG. 19C, processing in which the partial images P are joined in a state in which the edge portions thereof are overlapped with each other is performed. In the case in which the projected lateral magnifications of the individual objective optical systems 11 are one, it is not necessary to perform such joining processing.

As has been described above, in the linear scanning observation apparatus 100 that acquires a two-dimensional image of the sample A by scanning the line sensor 13 with respect to the sample A, employing oblique illumination affords an advantage in that it is possible to acquire high-contrast images G even with colorless, transparent phase objects, such as cells. In addition, by utilizing the top plate 1a of the vessel as a reflective member and by consolidating all of the illumination portion 3, the image acquisition portion 4, the focus adjusting mechanism 5, and the scanning mechanism 6 below the stage 2, there is an advantage in that it is possible to realize a compact apparatus.

Furthermore, because all of the illumination portion 3, the image acquisition portion 4, the focus adjusting mechanism 5, and the scanning mechanism 6 are accommodated below the stage 2 in an airtight state in the housing, it is possible to accommodate the apparatus inside a high-temperature, high-humidity incubator, and thus, it is possible to acquire the images G over time while culturing the sample A in the incubator.

In addition, as a result of disposing the prism 10 in the vicinity of the objective optical system group 12, it is possible to cope with a vessel 1 having the top plate 1a at a low position.

In other words, in order to satisfy Conditional Expressions (1)-(4), described above, in the case in which a vessel 1 having the top plate 1a at a low position is used, it is necessary to bring the position at which the illumination light is emitted from the illumination portion 3 close to the optical axis of the objective optical system group 12. However, due to interference by lenses, frames, or the like of the objective optical system group 12, it is difficult to dispose the line light source 8 in the vicinity of the objective optical system group 12.

Therefore, as shown in FIG. 16, the prism 10 is inserted between the mounting base 2a and the objective optical system group 12 to dispose the prism 10 above the objective optical system group 12 and at a position that is slightly shifted in a radial direction from the optical axis, and thus, the line light source 8 is disposed at a position that is separated from the objective optical system group 12 in a horizontal direction. By doing so, it is possible to emit the illumination light obliquely upward from the vicinity of the optical axis of the objective optical system group 12.

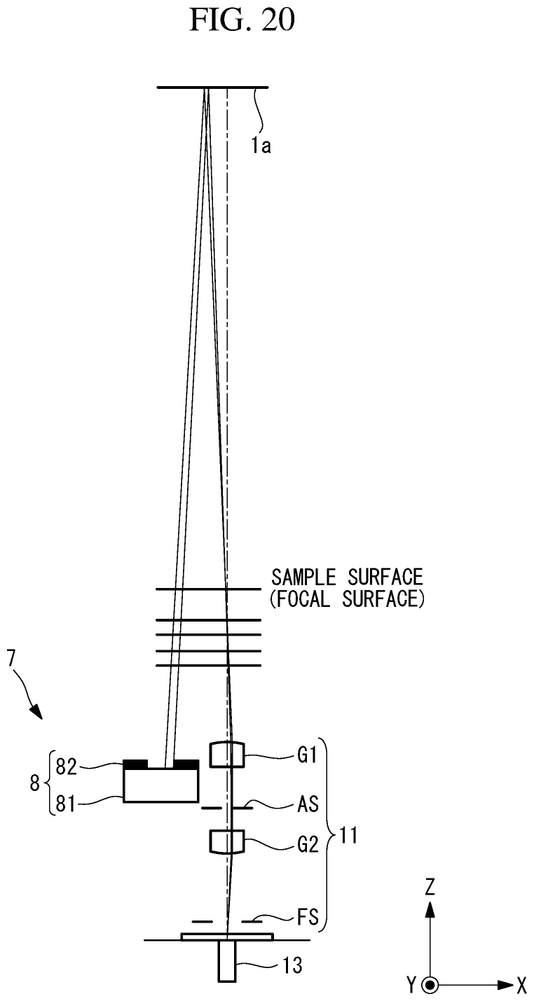

In the case in which a vessel 1 having the top plate 1a at a high position is used, in order to obtain an optical image of the sample A, the image having a contrast created by oblique illumination, the illumination light is emitted obliquely upward from the position separated from the optical axis of the objective optical system group 12. Therefore, as shown in FIG. 20, the prism 10 may be omitted, and the line light source 8 may be disposed at a position at which the illumination light is emitted obliquely upward from the line light source 8.

Furthermore, in the case in which only vessels 1 in which the heights of the top plates 1a are the same are used, the relationship among relative positions of the sample surface, the reflective surface (top plate 1a) of the reflective member, and the illumination optical system 7 does not change, and the angle at which the illumination light is radiated onto the sample A becomes constant. Therefore, in this case, the prism 10 and the cylindrical lens 9 may be omitted, as shown in FIG. 20.

Although the top plate 1a of the vessel 1 is utilized as a reflective member for reflecting the illumination light, alternatively, a system in which the illumination light is reflected by a reflective member provided above the vessel 1 may be employed.

In addition, in this embodiment, the display portion displays the proliferation speeds in association with the positional information by means of color-coding superimposed on the images G; however, alternatively, the positional information and the proliferation speeds may be displayed in association with each other by using values.

In addition, in this embodiment, sorting of the colonies Y based on combinations of information obtained by measuring and calculating the height dimensions of the colonies Y may be presented to the user. By doing so, it is possible to provide the user with more precise information related to sorting of the colonies Y.

In addition, in this embodiment, an apparatus that captures images in a line shape has been described as an example of the observation apparatus 100; however, alternatively, an apparatus that captures images in a square shape may be employed.

As a result, the above-described embodiment leads to the following aspects.

An aspect of the present invention is directed to a cell-image processing apparatus including: a measurement-region extracting portion that extracts, regarding a plurality of images acquired by capturing, over time, images of cells that are being cultured, a plurality of common measurement regions among the images; a proliferation-speed calculating portion that calculates proliferation speeds of the cells contained in the respective measurement regions extracted by the measurement-region extracting portion; and a storage portion that stores the proliferation speeds calculated by the proliferation-speed calculating portion and positional information of the respective measurement regions in association with each other.

With this aspect, when the plurality of images are input, the measurement-region extracting portion extracts the plurality of common measurement regions among the respective images. Then, the proliferation-speed calculating portion calculates the proliferation speeds which indicate increases/decreases in the number of cells per unit time in the common measurement regions in two images acquired at times that are separated by a time interval. The storage portion stores the proliferation speeds calculated in this way in association with the positional information of the measurement regions corresponding to said proliferation speeds.

Accordingly, when an observer manually specifies or the apparatus automatically specifies one of the measurement regions in one of the images, it is possible to recognize measurement regions having proliferation speeds that are close to the proliferation speed of the cells in said measurement region.

Specifically, in the case in which the observer performs observation by determining, while viewing the image, that a measurement region is the measurement region in which specific cells, for example, pluripotent cells, are present, it is possible to extract, in a simple manner, other measurement regions having proliferation speeds similar to that in said measurement region. In other words, it is possible to perform observation by selecting cells having specific proliferation speeds, and thus, it is possible to efficiently extract specific cells that are present in a culture vessel regardless of the sizes of colonies.

The above-described aspect may include a display portion that displays the proliferation speeds and the positional information of the measurement regions stored in the storage portion in association with each other in a manner in which the two types of information are associated with each other.

With this configuration, it is possible to perform observation by selecting the measurement regions in a simple manner on the basis of the proliferation speeds displayed on the display portion.

In addition, in the above-described aspect, the storage portion may store the measurement regions by classifying the measurement regions into a plurality of groups in accordance with the proliferation speeds calculated by the proliferation-speed calculating portion.

With this configuration, it is possible to specify measurement regions belonging to the same groups in a simple manner, and thus, it is possible to easily perform observation.

In addition, in the above-described aspect, the display portion may display one of the images, and displays the measurement regions by color-coding each of the groups.

With this configuration, it is possible to specify the measurement regions belonging to the same groups in a simple manner on the basis of the colors, and thus, it is possible to easily perform observation.

In addition, in the above-described aspect, the display portion may display the measurement regions by color-coding the measurement regions by using colors in accordance with the proliferation speeds.

With this configuration, it is possible to display the measurement regions by means of a heat map, and thus, it is possible to precisely sort the cells by facilitating recognition of differences in the proliferation speeds.

In addition, another aspect of the present invention is directed to a cell-image processing apparatus including: a processor; and a memory, wherein, regarding a plurality of images acquired by capturing, over time, images of cells that are being cultured, the processor extracts a plurality of common measurement regions among the images, and calculates proliferation speeds of the cells contained in the respective extracted measurement regions, and the memory stores the calculated proliferation speeds and positional information of the respective measurement regions in association with each other.

The present invention affords an advantage in that it is possible to efficiently extract specific cells that are present in a culture vessel.

### REFERENCE SIGNS LIST

51 cell-image processing apparatus
52 image storage portion (memory)
53 measurement-region extracting portion
54 proliferation-speed calculating portion (processor)
55 information storage portion (memory, storage portion)
A sample (cell)
G image
R measurement region
X cell

1. A cell-image processing apparatus comprising:
a processor comprising hardware; and
a storage,
wherein the processor is configured to:
extract, regarding a plurality of images acquired by capturing, over time, images of cells that are being cultured, a plurality of common measurement regions among the images; and
calculate proliferation speeds of the cells contained in the respective extracted measurement regions, and
wherein the storage is configured to store the calculated proliferation speeds and positional information of the respective measurement regions in association with each other.

2. The cell-image processing apparatus according to claim 1, wherein the extracting of the plurality of common measurement regions recognizes boundaries between cells or colonies and other areas, and extracts the measurement regions on a basis of the recognized boundaries.

3. The cell-image processing apparatus according to claim 2, wherein the extracting of the plurality of common measurement regions recognizes boundaries between colonies and other areas, and extracts the recognized colonies as the measurement regions.

4. The cell-image processing apparatus according to claim 2, wherein the calculating of the proliferation speeds calculates the proliferation speeds on a basis of areas of the colonies.

5. The cell-image processing apparatus according to claim 2, wherein the extracting of the plurality of common measurement regions sets the measurement regions assuming that a plurality of cells or colonies that are close to each other are in same regions.

6. The cell-image processing apparatus according to claim 3, wherein the processor is further configured to display a video image indicating changes over time in the measurement regions containing the colonies.

7. The cell-image processing apparatus according to claim 6, wherein the processor is further configured to calculate center positions of the colonies in the measurement regions and set the center positions to be a center of the video image.

8. The cell-image processing apparatus according to claim 1, further comprising:
a display that displays the proliferation speeds and the positional information of the measurement regions stored in the storage in association with each other in a manner in which the proliferation speeds and the positional information are associated with each other.

9. The cell-image processing apparatus according to claim 8, wherein the storage stores the measurement regions by classifying the measurement regions into a plurality of groups in accordance with the calculated proliferation speeds.

10. The cell-image processing apparatus according to claim 9, wherein the display displays one of the images, and displays the measurement regions by color-coding each of the groups.

11. The cell-image processing apparatus according to claim 8, wherein the display displays the measurement regions by color-coding the measurement regions by using colors in accordance with the proliferation speeds.

12. The cell-image processing apparatus according to claim 11, wherein the display displays one of the plurality of images, and displays, so as to be superimposed on the image, the measurement regions by using color-coding in accordance with the proliferation speeds.

13. The cell-image processing apparatus according to claim 11, wherein the display displays the measurement regions by using color-coding in accordance with the proliferation speeds without using superimposed display on one of the plurality of images.

14. The cell-image processing apparatus according to claim 8, wherein the display displays, in response to one of the measurement regions being specified, a graph indicating a change over time in the proliferation speed in the specified region.

15. The cell-image processing apparatus according to claim 8, wherein the display displays a video image showing a change over time in the specified measurement region.
